(19) United States
(12) Patent Application Publication
(10) Pub. No.: US 2026/0240451 A1
(43) Pub. Date: Aug. 20, 2026

(54) GAIT CORRECTION MANAGEMENT TERMINAL, GAIT CORRECTION SYSTEM, AND GAIT CORRECTION MANAGEMENT METHOD

(71) Applicant: SOFT SENSE LIFE TECH PTE. LTD., SINGAPORE (SG)

(72) Inventor: KUAN-CHIEN CHOU, Taipei City (TW)

(21) Appl. No.: 19/228,397

(22) Filed: Jun. 4, 2025

(30) Foreign Application Priority Data
Feb. 14, 2025 (CN) 202510163314.1

(51) Int. Cl. A61B 5/103; A43B 3/44

(52) U.S. Cl. CPC A61B 5/1038; A43B 3/44

(57) ABSTRACT

The present invention discloses a gait correction management method, which is used by a gait detection unit, a gait correction unit, and a processing module through cooperation. The gait correction unit is connected to the gait detection unit. The gait correction management method includes the following steps: generating gait information based on a plurality of pressure signals output by a pressure detection module of the gait detection unit; generating, by a processing module, gait correction configuration information based on the gait information and content in a gait database; and adjusting thicknesses, a quantity of layers, and/or a softness/hardness parameter of the plurality of elastic correction blocks of the gait correction unit based on the gait correction configuration information. In addition, the present invention further provides a gait correction management terminal and a gait correction system.

This Non-provisional application claims priority under 35 U.S.C. §119(a) on Patent Application No. 202510163314.1 filed in People's Republic of China on February 14, 2025, the entire contents of which are hereby incorporated by reference.

### FIELD OF THE INVENTION

The present invention relates to the field of the gait correction technology, and in particular to a gait correction management terminal, a gait correction system, and a gait correction management method.

### BACKGROUND OF THE INVENTION

In modern society, many individuals frequently suffer from foot-related issues, such as arch deformities, flat feet, overpronation, supination, or plantar fasciitis. These problems are usually alleviated or improved through the use of corrective insoles.

In existing technologies, a process of producing the corrective insoles typically requires a series of lengthy and complex procedures, including medical evaluation, foot shape measurement, corrective insole production, trial fitting, and adjustment. First, patients are required to undergo evaluation by physicians or physical therapists to determine foot problems and corresponding needs, which may include examinations of skeleton, muscles, and joints of the feet. Next, professionals perform foot measurements, including a foot length, a foot width, a foot arch height, and the like. Based on results of the medical evaluation and foot measurements, appropriate corrective insoles are either custom-made or selected by the professionals. A patient then tries on the insoles to ensure proper fit, and adjustments are made as necessary to achieve optimal effectiveness.

However, correction cannot rely solely on the corrective insoles. The patient needs to undergo regular checkup with medical professionals after wearing the corrective insoles for a period of time. As foot shapes gradually improve, new insoles need to be made. This leads to drawbacks of time-consuming inspection and production, as well as the inability to immediately inspect correction effect.

Therefore, providing a gait correction management terminal, a gait correction system, and a gait correction management method that can improve the above problems is currently one of important issues.

### SUMMARY OF THE INVENTION

In view of this, the present invention is intended to provide a gait correction management terminal, a gait correction system, and a gait correction management method to reduce the inconvenience of repeated inspections and improve correction efficiency.

In addition, the present invention is further intended to provide a gait correction management terminal, a gait correction system, and a gait correction management method, which can quickly and easily produce and update corrective insoles, thereby reducing the time for patients to wait for insole production and lowering production costs.

To achieve the above purposes, the present invention provides a gait correction system, including a gait detection unit, a gait correction unit, and a gait correction management terminal. The gait detection unit is provided with an insole, a pressure detection module, and a communication processing module. The insole is provided with a first side and a second side that are opposite to each other, and the pressure detection module is disposed on the first side and electrically connected to the communication processing module. The communication processing module receives a plurality of pressure signals output by the pressure detection module and generates gait information accordingly. The gait correction unit is provided with a plurality of elastic correction blocks, which are disposed on the second side of the insole. The gait correction management terminal is provided with a communication module, a processing module, and a gait database coupled to each other. The processing module generates gait correction configuration information based on the gait information and content in a gait database. The gait correction configuration information includes adjustment information of thicknesses, a quantity of layers, and/or a softness/hardness parameter of the plurality of elastic correction blocks of the gait correction unit.

In an embodiment of the present invention, a plurality of stacked elastic layers are provided for one of the plurality of elastic correction blocks of the gait correction unit, and the plurality of elastic layers are added, removed, and/or adjusted based on the gait correction configuration information.

In an embodiment of the present invention, the insole is provided with a bottom layer and a top layer in sequence. The pressure detection module is located between the bottom layer and the top layer, and the plurality of elastic correction blocks are connected to one side of the bottom layer relative to the top layer.

In an embodiment of the present invention, the pressure detection module is provided with an elastic film and a plurality of pressure sensors. The plurality of pressure sensors and conductive lines thereof are printed on a surface of the elastic film.

In an embodiment of the present invention, the plurality of conductive lines are electrically connected to the communication processing module through a flexible circuit board.

In addition, to achieve the above purpose, the present invention further provides a gait correction management terminal, configured to communicate with a gait detection unit and cooperate with a gait correction unit for application. The gait correction management terminal includes a gait database, a communication module, and a processing module. The communication module receives gait information sent by the gait detection unit. The processing module is coupled to the gait database and the communication module separately, and generates gait correction configuration information based on the gait information and content in the gait database. The gait correction configuration information includes adjustment information of thicknesses, a quantity of layers, and/or a softness/hardness parameter of the plurality of elastic correction blocks provided for the gait correction unit.

In an embodiment of the present invention, the gait correction configuration information generated by the processing module is recorded in the gait database and applied to a subsequent calculation.

In addition, to achieve the above purposes, the present invention further provides a gait correction management method, which is used by a gait detection unit, a gait correction unit, and a processing module through cooperation. The gait correction unit is connected to the gait detection unit. The gait correction management method includes the following steps: generating gait information based on a plurality of pressure signals output by a pressure detection module of the gait detection unit; generating, by a processing module, gait correction configuration information based on the gait information and content in a gait database; and adjusting thicknesses, a quantity of layers, and/or a softness/hardness parameter of the plurality of elastic correction blocks of the gait correction unit based on the gait correction configuration information.

Based on the above, the present invention discloses the gait correction management terminal, the gait correction system, and the gait correction management method, through the technologies, the gait detection unit and the gait correction unit are embedded into a shoe daily worn by a user under gait correction, and gait information of the user under gait correction is measured in real time and recorded continuously. Such gait information is transmitted to the gait correction management terminal, and after comprehensive calculation, the plurality of elastic correction blocks are configured in the gait correction unit. Finally, the gait correction configuration information is generated based on calculation results and an existing elastic correction block configuration for a user under gait correction. The gait correction configuration information includes adjustment information of a quantity of layers and/or a softness/hardness parameter of the elastic correction blocks. In other words, the user under gait correction only needs to update some elastic correction blocks to continue to use the insole, achieving the effect of continuous correction. Therefore, the user under gait correction neither needs to spend a lot of money to reproduce corrective insoles, nor has to wait for a long production process.

### BRIEF DESCRIPTION OF THE DRAWINGS

FIG. 1 is a schematic block diagram of a gait correction system according to a preferred embodiment of the present invention.

FIG. 2 is a schematic structural diagram of a gait detection unit according to a preferred embodiment of the present invention.

FIG. 3 is a schematic structural diagram of a pressure detection module according to a preferred embodiment of the present invention.

FIG. 4 is a schematic structural diagram of another pressure detection module.

FIG. 5 is a schematic structural diagram of a gait correction unit according to a preferred embodiment of the present invention.

FIG. 6 is a flowchart of a gait correction management method according to a preferred embodiment of the present invention.

### DETAILED DESCRIPTION OF PREFERRED EMBODIMENTS

The following clearly and completely describes technical solutions in embodiments of the present invention with reference to accompanying drawings in embodiments of the present invention. Many specific details are set forth in the following description to fully understand the present invention. However, the present invention may alternatively be implemented in other ways different from those described herein. Those skilled in the art can make similar promotions without violating the connotation of the present invention. Therefore, the present invention is not limited to specific embodiments disclosed below.

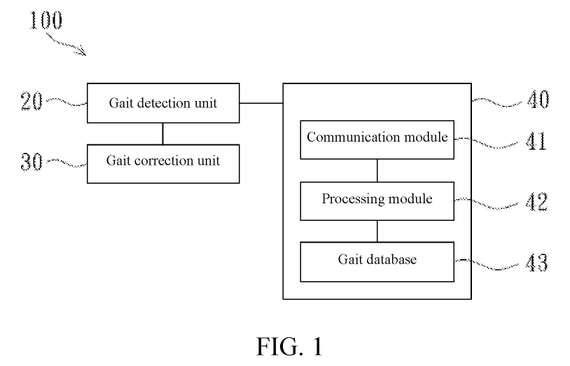

Refer to FIG. 1. A gait correction system 100 in a preferred embodiment of the present invention includes a gait detection unit 20, a gait correction unit 30, and a gait correction management terminal 40. The gait detection unit 20 is provided with an insole 21, a pressure detection module 22, and a communication processing module 23.

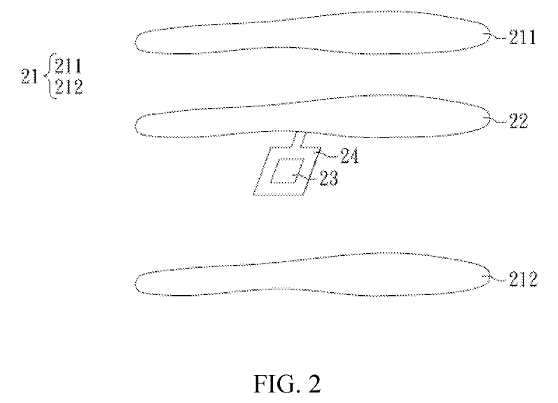

The insole 21 is configured to be installed inside a shoe to accommodate a user's foot and provide cushioning for the foot. As shown in FIG. 2, the insole 21 is provided with a top layer 211 and a bottom layer 212 arranged opposite to each other. The top layer 211 is configured to contact the user's foot, while the bottom layer 212 is configured to contact a bottom inside the shoe. The top layer 211 may be made of fabric, such as but not limited to cotton, polyester fiber, nylon, or the like. The bottom layer 212 may be made of a polymer material, such as but not limited to EVA including thermoplastic polymers, PU or PORON including polyurethane. In addition, in this embodiment, for ease of description, a side of the insole 21 near the top layer 211 may be referred to as a first side, and a side near the bottom layer 212 may be referred to as a second side.

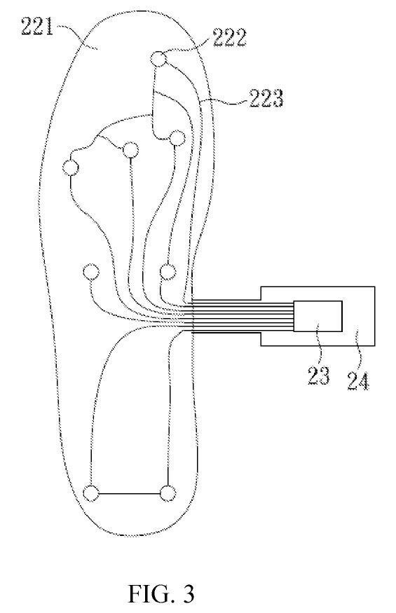

The pressure detection module 22 is disposed between the top layer 211 and the bottom layer 212 of the insole 21, and is provided with an elastic film 221 and a plurality of pressure sensors 222. As shown in FIG. 3, the pressure sensors 222 are disposed on a surface of the elastic film 221. For the sake of simplicity and readability, each circle in FIG. 3 represents one pressure sensor and is not labeled one by one. Therefore, in this embodiment, for example, there are eight pressure sensors 222 in the pressure detection module 22. The pressure sensors 222 may be piezoelectric sensors that utilize piezoelectric effect, and of course, pressure sensors such as but not limited to resistive, capacitive, magneto resistive, or optical sensors can alternatively be used. In other embodiments, the pressure sensors 222 may also be formed on the surface of the elastic film 221 through 3D printing or inkjet printing. In addition, conductive lines 223 of the pressure sensors 222 may alternatively be formed on the surface of the elastic film 221 by printing.

The elastic film 221 has flexibility and waterproof or water repellent properties, and is made of a material including but not limited to silicone, polyurethane, or thermoplastic polyurethane. It is worth mentioning that the pressure detection module 22 may alternatively include another waterproof elastic film (not shown in the figure), which can be combined with the elastic film 221 to wrap the pressure sensors 222 in two layers of the elastic film, thereby enhancing the waterproof and moisture-proof properties.

As shown in FIG. 2, the pressure sensors 222 of the pressure detection module 22 detect a pressure from the top layer 211 of the insole 21. In other words, the pressure detection module 22 generates a pressure signal based on a pressure applied by a user's foot to the pressure sensors 222.

The communication processing module 23 is electrically connected to the conductive lines 223 on the elastic film 221 through a flexible circuit board 24. It is worth mentioning that in this embodiment, the communication processing module 23 is a system on chip (SoC), which may include but is not limited to a microprocessor, a wireless communication module, an inertial measurement unit (IMU), a memory, an input/output interface, and other sub modules. The wireless communication module may include, but is not limited to, a Wi-Fi module, a Bluetooth module, or a near field communication (NFC) module. The communication processing module 23 receives a plurality of pressure signals output by the pressure detection module 22 and generates gait information accordingly. The microprocessor of the communication processing module 23 can generate various pieces of gait information related to the user's foot based on time and signal sizes of the plurality of pressure signals, including but not limited to a center of pressure (COP) trajectory, a ground reaction force (GRF), and a butterfly diagram. In addition, it is worth mentioning that the communication processing module 23 can be configured in a body (not shown in the figure) of a shoe, such as the bottom or quarter of the shoe.

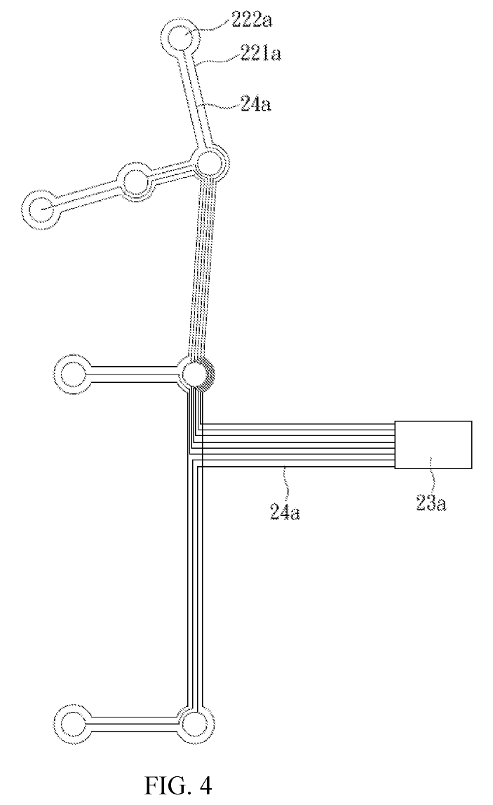

FIG. 4 shows another implementation of the pressure detection module. Unlike FIG. 3, a shape of the elastic film 221a in FIG. 4 is tailored to configuration of the pressure sensors 222a, and a part of the flexible circuit board 24a is also provided on the elastic film 221a to electrically connect each pressure sensor 222a to the communication processing module 23a. In this embodiment, for example, there are eight pressure sensors 222a.

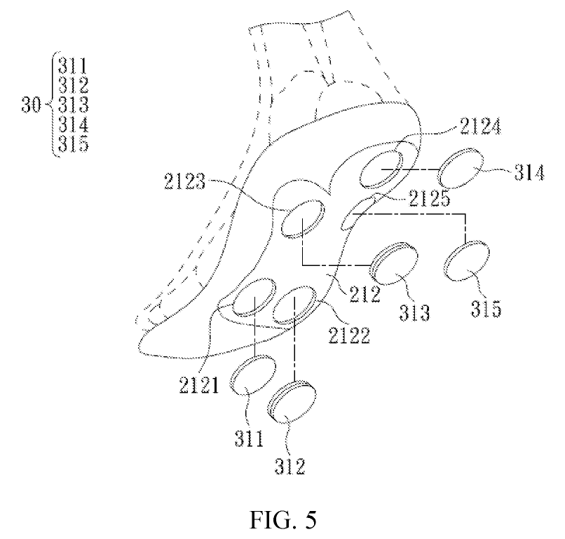

As shown in FIG. 5, the gait correction unit 30 is provided with a plurality of elastic correction blocks 311–315, which are disposed on a side of the bottom layer 212 of the insole 21 away from the top layer 211. The elastic correction blocks 311–315 are distributed on the bottom layer 212, for example, elastic correction blocks 311 and 312 are configured corresponding to toes, an elastic correction block 313 is configured corresponding to an arch of the foot, and elastic correction blocks 314 and 315 are configured corresponding to a heel. A quantity and placement of the elastic correction blocks are not restrictive, and design can be adjusted adaptively. In this embodiment, a material of the elastic correction blocks 311–315 may include but is limited to silicone, polyurethane, EVA (ethylene vinyl acetate copolymer), memory foam, thermoplastic elastomer (TPE), or cork.

In this embodiment, a surface of the bottom layer 212 of the insole 21 may be provided with a plurality of grooves 2121–2125 corresponding to the elastic correction blocks 311–315, and the elastic correction blocks 311–315 and the grooves 2121–2125 can be combined through snap–fit design, hook and loop fasteners, a sliding mechanism, or a pin and hole mechanism. In other words, the elastic correction blocks 311–315 can be easily combined with or separated from the grooves 2121–2125, to facilitate the update of different elastic correction blocks.

Taking a mortise and tenon structure as an example, corresponding mortises and grooves can be designed on the elastic correction blocks and grooves, so that the elastic correction blocks can be accurately embedded in the grooves. In addition, it is necessary to ensure that the mortises and tenons are made of a material with sufficient elasticity for easy clamping and disassembly, while firmness is maintained. In addition, taking a pin structure as an example, corresponding pins and holes can be designed on the elastic correction blocks and grooves, so that the pins can be fastened in the holes.

It is worth mentioning that the elastic correction blocks can also be combined with each other through the above structure. In other words, elastic correction blocks corresponding to a same groove may include a plurality of layers of elastic correction blocks combined with each other. In addition, the elastic correction blocks can be combined with each other through hot pressing in addition to the above structure.

Still refer to FIG. 1. The gait correction management terminal 40 is provided with a communication module 41, a processing module 42, and a gait database 43 coupled to each other. The gait correction management terminal 40 may be a multi-entity infrastructure based on cloud services. In this embodiment, only the communication module 41, processing module 42, and gait database 43 as a basis are used as an example.

The communication module 41 is capable of receiving gait information generated by the communication processing module 23 and transmitting the gait information to the processing module 42. In addition, the gait information generated by the communication processing module 23 may also be input to the gait correction management terminal 40 through various forms of memory apparatuses, such as a portable memory.

The processing module 42 generates gait correction configuration information based on the gait information generated by the communication processing module 23 and content recorded in the gait database 43. The gait correction configuration information includes adjustment information of thicknesses, a quantity of layers, and/or a softness/hardness parameter of the plurality of elastic correction blocks 311–315 of the gait correction unit 30. It is worth mentioning that the processing module 42 can also calculate a step distance, a step frequency, a height of the foot after leaving the ground, an angle of the foot between leaving the ground and landing, impact force during landing based on an artificial intelligence calculation engine, biomechanics, sports science, and prescription, and in combination with analysis of a COP trajectory, a ground reaction force, and a butterfly diagram, to estimate various pieces of data such as a hip joint, a knee joint, and an ankle joint status.

In this embodiment, the gait information is generated by the gait detection unit 20. When an object of the information is use of the gait detection unit 20 for the first time, the processing module 42 can calculate gait correction configuration information including the thicknesses, a quantity of layers, and/or a softness/hardness parameter of the plurality of elastic correction blocks 311–315 of the gait correction unit 30 based on general information recorded in the gait database 43 and the gait information. Next, the gait correction configuration information is transmitted to a specific terminal through the communication module 41, such as a computer terminal of a hospital, a computer terminal of a manufacturer, and/or a communication apparatus of a user. Subsequently, a corresponding gait correction unit 30 can be generated based on the gait correction configuration information.

In addition, when the object of the information is a user who has previously used the gait correction system 100, it means that the gait database 43 records the user's previous course data, the processing module performs calculation based on the course data recorded in the gait database 43 and the gait information, to generate adjustment information of gait correction configuration information including the thicknesses, the quantity of layers, and/or a softness/hardness parameter of the plurality of elastic correction blocks 311–315 of the gait correction unit 30. Similarly, the gait correction configuration information with the adjustment information can also be transmitted to a specific terminal through the communication module 41, and subsequently, the configuration of the elastic correction block of the gait correction unit 30 can be adjusted based on the gait correction configuration information.

It is worth mentioning that stacked elastic layers can be easily added to the elastic correction block of the gait correction unit, and a plurality of elastic layers are added, removed, and/or adjusted based on the gait correction configuration information. In other words, in this embodiment, after receiving the gait correction configuration information, the user, hospital, or manufacturer can directly add other elastic layers to original elastic correction blocks or remove an unnecessary elastic layer. Therefore, the user does not need to produce a new corrective insole, which wastes additional costs and time, and material waste and environmental burden can also be prevented.

A mortise and tenon structure, a hook and loop structure, or a sliding buckle or pin structure can be used between elastic layers, and a combined structure and the elastic layers can be made of the same material and formed as a whole. In addition, in other embodiments, thermal compression bonding can also be used between the elastic layers.

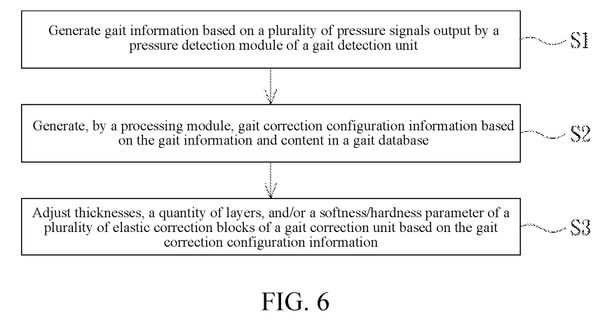

Next, refer to FIG. 6. The gait correction management method in a preferred embodiment of the present invention is applied to the above gait correction system 100. The gait correction management method mainly includes step S1 to step S3.

Step S1: Generate gait information based on a plurality of pressure signals output by a pressure detection module 22 of a gait detection unit 20. The gait information includes a COP trajectory, a GRF, and a butterfly diagram. In other words, the gait information is dynamic information related to user's walking or running. Step S2: Generate, by a processing module 42 of the gait correction management terminal 40, gait correction configuration information based on the gait information and content in a gait database 43. Step S3: Adjust thicknesses, a quantity of layers, and/or a softness/or hardness parameter of elastic correction blocks of a gait correction unit 30 based on the gait correction configuration information. Therefore, the gait correction management method has been explained above and is not further elaborated.

In summary, in the gait correction management terminal, the gait correction system, and the gait correction management method disclosed in the present invention, through the technologies, the gait detection unit and the gait correction unit are embedded into a shoe daily worn by a user under gait correction, and gait information of the user under gait correction is measured in real time and recorded continuously. Such gait information is transmitted to the gait correction management terminal, and after comprehensive calculation, the plurality of elastic correction blocks are configured in the gait correction unit. Finally, the gait correction configuration information is generated based on a difference between calculation results and an existing elastic correction block configuration of the user. The gait correction configuration information includes adjustment information of thicknesses, a quantity of layers, and/or a softness/hardness parameter. In other words, the user no longer needs to waste time frequently scheduling tests, and only needs to update some elastic correction blocks for the corrective insole to continue to use the insole, achieving the effect of continuous correction. Therefore, the user neither needs to spend a lot of money to reproduce the corrective insole, nor has to wait for a long production process. Medical institutions can also immediately know a user's correction status and assess a time point for updating the elastic correction block at any time.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A gait correction system, comprising:
a gait detection unit, comprising an insole, a pressure detection module, and a communication processing module, wherein the insole is provided with a first side and a second side that are opposite to each other, the pressure detection module is disposed on the first side and electrically connected to the communication processing module, the communication processing module receives a plurality of pressure signals output by the pressure detection module and generates gait information accordingly;
a gait correction unit, comprising a plurality of elastic correction blocks, wherein the elastic correction blocks are disposed on the second side of the insole; and
a gait correction management terminal, comprising a processing module, a communication module, and a gait database that are mutually coupled, wherein the processing module generates gait correction configuration information based on the gait information and content in the gait database,
wherein the gait correction configuration information comprises adjustment information of thicknesses, a quantity of layers, and/or a softness/hardness parameter of the plurality of elastic correction blocks of the gait correction unit.

2. The gait correction system according to claim 1, wherein a plurality of stacked elastic layers are provided for one of the plurality of elastic correction blocks of the gait correction unit, and the plurality of elastic layers are added, removed, and/or adjusted based on the gait correction configuration information.

3. The gait correction system according to claim 1, wherein the insole is provided with a bottom layer and a top layer in sequence, wherein the pressure detection module is located between the bottom layer and the top layer, and the plurality of elastic correction blocks are connected to one side of the bottom layer relative to the top layer.

4. The gait correction system according to claim 3, wherein the pressure detection module is provided with an elastic film and a plurality of pressure sensors, and the plurality of pressure sensors and conductive lines thereof are printed on the elastic film.

5. The gait correction system according to claim 4, wherein the plurality of conductive lines are electrically connected to the communication processing module through a flexible circuit board.

6. A gait correction management terminal, configured to communicate with a gait detection unit and cooperate with a gait correction unit for application, comprising:
a gait database;
a communication module, receiving gait information sent by the gait detection unit; and
a processing module, coupled to the gait database and the communication module separately, and generating gait correction configuration information based on the gait information and content in the gait database,
wherein the gait correction configuration information comprises adjustment information of thicknesses, a quantity of layers, and/or a softness/hardness parameter of the plurality of elastic correction blocks provided for the gait correction unit.

7. The gait correction management terminal according to claim 6, wherein the gait correction configuration information generated by the processing module is recorded in the gait database and applied to a subsequent calculation.

8. A gait correction management method, used by a gait detection unit, a gait correction unit, and a gait correction management terminal through cooperation, wherein the gait correction unit is connected to the gait detection unit, and comprising:
generating gait information based on a plurality of pressure signals output by a pressure detection module of the gait detection unit;
generating, by a processing module of the gait correction management terminal, gait correction configuration information based on the gait information and content in a gait database; and
adjusting thicknesses, a quantity of layers, and/or a softness/hardness parameter of the plurality of elastic correction blocks of the gait correction unit based on the gait correction configuration information.

9. The gait correction management method according to claim 8, wherein a plurality of stacked elastic layers are provided for one of the plurality of elastic correction blocks, and the plurality of elastic layers are added, removed, and/or adjusted based on the gait correction configuration information.

10. The gait correction management method according to claim 8, wherein the gait correction configuration information generated by the processing module is recorded in the gait database and applied to a subsequent calculation.
